(19) United States
(12) Patent Application Publication
(10) Pub. No.: US 2012/0094372 A1
(43) Pub. Date: Apr. 19, 2012

(54) Ex Vivo Cell Culture: Enabling Process and Devices

(76) Inventors: Ashok Mukhopadhyay, Kolkata (IN); Aroop Kumar Dutta, Lucknow (IN); Ranjna Dutta, Lucknow (IN)

(21) Appl. No.: 12/906,512

(22) Filed: Oct. 18, 2010

(51) Int. Cl. C12M 3/06; C12M 3/00

(52) U.S. Cl. 435/297.1; 435/289.1

(57) ABSTRACT

A device which is capable of providing an artificial condition for three dimensional culture of animal and human cells comprising of two components; First component is a vessel coated in the entire internal surface rendered not suitable for cell adhesion and proliferation and a second component is a cell interactive biomaterial on which cells can attach and grow as monolayer or multiple layers in three dimensions.

### FIELD OF INVENTION

The present invention relates to a device which is capable of providing an artificial condition for culture of animal and human cells in three dimensional (3D), tissues and other cell culture formats by recreating the appropriate environment similar to the animal or human body.

### BACKGROUND OF THE INVENTION

Technical requirements for recreating ideal cell culture conditions similar to in vivo condition prevailing in an animal or human body have been addressed here. Conventionally, cells are cultured as a two dimensional layer, called monolayer, in presence of blood derived serum containing liquid nutrient media, commonly on flat plastic or glass surfaces. This situation is in contrast with the cells in their natural environment of animal/human body, where they are growing as three dimensional multilayers without serum or artificial surface of plastic or glass.

The requirement of growth factors in a natural tissue is met through cooperation of other cells and organs. Surface for the cell culture in organs and tissues are provided by extra cellular matrix which is a network of collagenous polymers, glycosaminoglycans, and many other proteins and factors that provide structural and biological functionality to the cells of the tissues and organs.

Without these requirements cells in monolayer cultures typically performed in glass or plastic vessels behave quite differently form the cells present in the body (Elsdale T, Bard J, Collagen substrate for studies on cell behavior, Journal of Cell Biology, Vol. 54, 1972, pp 626-637). Therefore, a meaningful replication of organ and tissue features artificially for our understanding of physiology of healthy or diseased tissues and synthetic tissues for therapeutic applications, we need to incorporate following requirements in our cell culture methods.
A. 3D scaffold similar to extracellular matrix
B. Biological interaction among cells
C. Tissue/organ homeostasis
D. Nutritional requirements
E. Oxygen requirement
F. Toxicity removal
G. Convenient formats for application feasibility

Further explanation of the background and prior art is provided below:
A. Three dimensional (3D) scaffold: There are many types of three dimensional scaffolds developed as prior art. These involve use of synthetic and natural polymeric materials (Francesco Pampaloni, The third dimension bridges the gap between cell culture and live tissue, Nature Reviews: Molecular cell biology, Vol 8 Oct. 2007 pp 839; Jungwoo Lee, Three-Dimensional Cell Culture Matrices: State of the Art, TISSUE ENGINEERING: Part B, Volume 14, Number 1, 2008; Joseph Jagur-Grodzinski, Polym. Adv. Technol. 2006; 17: 395-418; Mano J F at al., J. R. Soc. Interface (2007) 4, 999-1030; Lutolf M P, NATURE BIOTECHNOLGY Vol 23 No 1 Jan. 2005).

Unfortunately, most of the 3D scaffold prepared form synthetic biomaterials do not exhibit interactivity with cells while natural biomaterials do not show desirable level of engineering properties. It is vital for a biomaterial to have both interactivity and engineering features for a practical 3D cell culture device for various applications development.
B. Tissue and organ homeostasis and biological interaction among cells can be achieved by co-culture methods. Typical devices used for culturing two different types of cells are culture inserts available form many manufacturers (e.g. Millipore, Nunc, Becton Dickinson etc.). It is not possible to culture more than two sets of cells using culture inserts as it is not possible to incorporate more than one inserts with one set of cells in each culture vessel containing other type of cell. It is also not possible to culture cells in three dimensional since such culture inserts do not incorporate a three dimensional scaffold as yet.
C. Nutritional requirements are met by adding culture medium containing specific nutritional molecules. When the nutrition is depleted over a few days the spent culture medium is replaced completely or partially with fresh culture medium. Alternatively, culture medium can be supplied and removed continuously, maintaining a constant nutritional level during the culture period. Continuous supply and removal of culture medium can be achieved by providing an inlet and outlet to the culture vessel through a pump or other means of flow of medium in and out of the culture vessel. Continuous removal and supplementation of nutrient medium termed as perfusion can also be done.
D. Interplay between various organs and tissues ensues physiological homeostasis. Biological factors produced from specific type of cell in tissue ensure survival and proper function maintenance of other type of cell in other tissues in vivo. Direct cell-cell contacts among variety of cells within an organ also provide signals for proper functioning. However, cells in culture do not have support from other types of cells, as cells normally under culture condition are all same type. At present 3D co-culture of more than two types of cell are not possible simply due to lack of proper hardwares. Some progress has been made recently (U.S. Pat. Nos. 7,186,548, 6,858,146, Albert P. Li, Drug Discovery Today, Vol 2 No. 2, 2005) allowing co-culture of multiple cell types albeit in monolayer (2D) format limiting their ability to perform as 3D tissues.
E. Oxygen requirement can be met through direct diffusion of oxygen to the culture medium from air-medium interface. However, due to poor solubility of oxygen in the medium and slow oxygen diffusion, it is only possible to supply sufficient oxygen to a small number of cells per unit volume of medium. Specific methods and devices have been developed to increase oxygen supply to cell culture or for medical purposes.
F. During the culture nutrients are consumed by the cells and toxic end products get accumulated in the medium that lead to inhibition of cell growth, unwanted differentiation and even cell death. Lactic acid and ammonia are major inhibitory and toxic substances that are accumulated by the consumption of glucose and glutamine. In addition to these unwanted substances other cell products also get accumulated in the medium leading to undesirable and unpredictable outcome in cell culture. Normally these toxic substances are removed from the culture medium by changing spent medium with fresh nutrient medium at regular intervals. Therefore, it is important to remove the spent medium time to time or continuously through perfusion as explained in the point C.
G. It is quite unlikely to culture cells as ex vivo equivalent of tissue or organ without these aspects incorporated in the devices. It is also important to keep the convenience of the process and devices for ex vivo cell culture from the methods, monitoring and analysis point of view as well, so that it is meaningful for application development.

Three dimensional cell cultures are performed using hydrogels like Matri-gel, Algi-matrix (Invitrogen), Me-Biol-gel (MeBiol Corp.), Hu-Bio-gel (Vivo Biosciences Inc.), Pura-metrix (3DM Inc.), Extra-cel (Glycosan Biosystems Inc.) and ECM Analog (ExCel Matrix Biological Devices P. Ltd.). While above methods or materials are suitable for a range of cell culture applications, the limitations of each method and material are numerous for a convenient device development for realistic applications.

### OBJECTS OF THE INVENTION

An object of this invention is to provide a device, which is capable of providing an artificial condition for there dimensional culture of animal and human cells, tissue and other cell culture formats;

Another object of this invention is to provide a device incorporating major requirements for the ex vivo cell culture and 3D scaffold in a range of conventional culture formats;

Still another object of this invention is to provide a device which allows a desired kind of cell in defined ECM environment by controlling the concentrations of ECM components in the 3D scaffold, in a culture vessel;

Yet another object of this invention is to provide a device which allows the culture of two or more types of cells in three dimensions in a device as convenient and conventional as Petri dish or other popular types of culture vessels;

Further object of this invention is to provide a device which allows creation of three dimensional artificial tissues by culturing desired types of cells using a three dimensional scaffold while incorporating other requirements for ex vivo culture mentioned above;

Still further object of this invention is to provide devices like Petri dishes, multiwell plates, T flasks etc., in innovative manner to render them suitable for three dimensional cell cultures for various applications.

### SUMMARY OF THE INVENTION

According to this invention there is a device which is capable of providing an artificial condition for three dimensional culture of animal and human cells comprising of two components;

First component is a vessel coated in the entire internal surface rendered not suitable for cell adhesion and proliferation and a second component is a cell interactive biomaterial on which cells can attach and grow as monolayer or multiple layers in three dimensions.

### BRIEF DESCRIPTION OF THE ACCOMPANYING DRAWINGS

FIG. 1: A device for 3D cell culture of animal/human cell for confocal/fluorescent microscopic study.

FIG. 2: A device for 3D cell culture.

FIG. 3: A device for 3D culture of two or more cell types.

FIGS. 4a, 4b & 4c: 3D cell growth in porous microcarrier

FIG. 4d: Cell viability on 3D scaffold.

FIG. 5: 3D cell growth of rat liver cells.

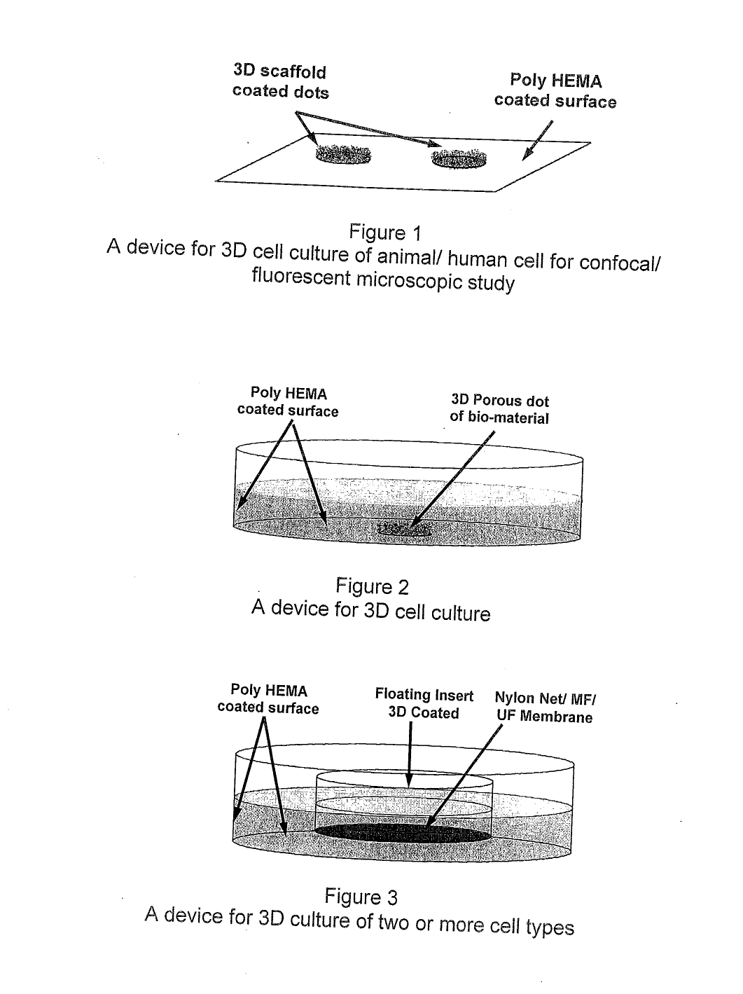

### ABBREVIATIONS AND EXPLANATION OF TECHNICAL TERMS

In vitro: In glass or artificial/lab environment.
In vivo: In a living system of an animal body.
Ex vivo: In an environment similar to a living system but arranged outside the animal body in an artificial/glass/laboratory environment.
3D: three dimensional.
2D: two dimensional.
Monolayer culture: A flat two dimensional layer of cells formed by growing cells on a flat plastic, glass or other suitable surfaces.
Co-culture: culture of two different types of cells in association with each other in a single vessel.

### DETAILED DESCRIPTION OF THE INVENTION

The occurrence of cell culture is diverse from purposes and formats point of views. Cell culture is preformed traditionally as two dimensional “monolayer” on the inner surface of Petri dishes, multiwell plates, T-flasks, bottles and on microcarrier surfaces.

The need for cell culture in three dimensions due to poor performance of cells in monolayer cultures have been highlighted in (Commentary: “Beyond the Petri dish”, Nature Biotechnology, Vol. 22, no. 2, 2004, 151-152; News Features: “Biology's New Dimension” Nature, Vol. 424, 2003, 870-872;

Editors: “Goodbye, Flat Biology?,” Nature, Vol. 424, 2003, 861). For example, when cells are cultured in a typical monolayer culture in any of the above formats cells are exposed to abnormal conditions not encountered in the body. Due to these abnormal conditions cells isolated from human or animal organs undergo a permanent change under monolayer culture condition and behave in undesirable manner.

It is desirable for the cells to behave in culture similar to their in vivo counterparts for understanding of normal and pathological tissue functioning, effects of drugs and tissue engineering applications.

It is known from the understanding of cell science that extracellular matrix (ECM) has profound effects on the cell behavior therefore, it is vital to incorporate ECM like cell interactive biomaterial in the cell culture devices in the form of 3D scaffolds while keeping the convenience of conventional cell culture devices for user friendly 3D cell culture solution.

There is a need for cell culture in three dimensions in specific formats of culture vessel for the following purposes.

| Formats | Purpose | Application Examples |
|---|---|---|
| Cover slip/slide | Confocal microscopy to | Cell differentiation |
| Slide Chamber | study cell function | biology research |
| Microchannels | Perfusion, long term culture | Cell behavior in |
| Slide/Chamber | | shear and long term |
| Multiwell plates | Cell growth/differentiation | Bioassays |
| Petri dishes | Cell growth/differentiation | Bioassays |
| Culture inserts | Cell-cell interaction/function | Bioassays |
| T flasks | Cell propagation | Quantitative cell yield |
| Roller bottle | Cell propagation | Quantitative cell yield |
| Microcarriers | Cell propagation | Large scale cell yield |

The invention provides a process using a porous three dimensional scaffold material with other provisions to create three dimensional tissues. It also provides a method of formation of devices for various cell culture requirements.

This invention provides the method of adaptation of conventional culture vessels to incorporate a variety of three dimensional scaffolds hence creating convenient devices for three dimensional cell cultures.

The process involves the 3D cell culture and tissue formation as disclosed in the U.S. patent application Ser. No. 12/171,910 using the cell-interactive material described therein.

In order to accomplish 3D cell culture and tissue formation of this invention two major components are required. First component is a culture vessel containing the second component. Second component is biomaterial providing support to cells for its proliferation and functioning.

The conventional cell culture vessels of first component are available in a variety of forms. For example, Petri dishes, multiwell plates, multiwell culture inserts, T flasks, roller bottles, spinner bottles etc. are developed for monolayer cultures offering convenient handling features.

This invention desires to incorporate traditionally familiar and convenient features of these culture vessels in an innovative manner and render them suitable for 3D cell culture instead of two dimensional monolayer cultures by incorporating 3D scaffold and other said ex vivo requirements.

The biomaterial for cell growth and function of the second component is a support material for cell growth. Biomaterial may be present in the culture vessel in the form of thin two dimensional coating or three dimensional scaffolds.

Three dimensional cell cultures are performed using 3D hydrogels scaffolds like Matri-gel, Algi-matrix (Invitrogen), Me-Biol-gel (MeBiol Corp.), Hu-Bio-gel (Vivo Biosciences Inc), Pura-metrix (3DM Inc.), and Extra-cel (Glycosan Biosystems Inc.) and ECM Analog (ExCel Matrix Biological Devices P. Ltd.).

3D cell culture and tissue formation can be performed using the cell-interactive material disclosed in the U.S. patent application Ser. No. 12/171,910.

3D cell culture and tissue formation can also be performed using 3D scaffold of synthetic biodegradable biomaterial like poly lactic acid, poly glycolic acid, poly caprolactone, poly vinyl alcohol, etc.

The invention allows the culture of more than two types of cells in three dimensions in a single device, as well as more than two types of biomaterial scaffold differing in their constituents both in qualitative forms and quantitative values in a single vessel. For example, desired kind of cells can be cultured in defined ECM environment by controlling the concentrations of ECM components in the 3D scaffold, in a culture vessel.

The 3D scaffold is created by the process and product as disclosed in U.S. patent application Ser. No. 12/171,910, as well as other methods known.

Cell culture can be performed on a 3D coating of biomaterial in the form of single or multiple dots varying in their ECM composition.

Tissue/organ homeostasis and biological interactions can be met through incorporating 3D scaffolds as discrete islands for cells of various organs to be cultured as 3D tissue. For example, a Petri dish can be coated with one or more type of 3D scaffolds as discrete dots for culturing cells of different organs or animal origin on each dot.

In order to prevent the cells growing on area other than dots (island), it is desirable to render the remaining surface of the Petri dish unsuitable for cell adhesion and culture as monolayer. This can be accomplished by coating the Petri dish by polyhydroxyethylmethacrylate (polyHEMA) (Folkman, J. and Moscona, A. (1978). Nature 273: 345-349.) and material as detailed in US patents (U.S. Pat. Nos. 5,977,257, 6,090,901).

The above Petri dish with 3D coated dots needs to be supplemented with sufficient nutrition media to support the cells growing as 3D tissue. If there is too much growth due to excess surface provided by the 3D scaffold then cells will not be supported nutritionally and will start dying in short time. Therefore, an optimal size of 3D dots is essential. Normally, a dot of 1-5 mm per 35 mm diameter Petri dish is sufficient to prevent cell over growth nutritionally and can support the cells on the 3D scaffold.

Oxygen requirement can be met by the surface diffusion to the culture medium. However, if the cell layers is thicker than 200 micron it would be difficult to support cells beyond such depths in the absence of convective flow of medium in the three dimensional scaffold. The limitation of oxygen diffusion can be met either by coating the 3D scaffold to a thickness less and 200 micron or by provision provided in the U.S. patent application Ser. No. 12/171,910. It is also possible to provide macro pores in 3D scaffold through which nutrition medium rich in oxygen can flow freely and provide sufficient oxygen to the cells growing in the interior of the scaffold.

Toxicity removal can be accomplished by removal of spent medium manually or continuously using a pump. Fresh medium can also be supplemented though a similar arrangement. It is essential to provide an inlet and outlet ports for medium to be exchanged from a reservoir using a pump.

3D scaffold and its support need to be preferably transparent for microscopic examination.

In another aspect of this invention is to device a cell culture insert coated with 3D scaffold that can be incorporated in a culture vessel in more than one number, hence allowing cells from more than one tissue type or organ to be co-cultured in a single vessel. Conventional co-culture devices can not be incorporated in more than one number in a culture vessel, therefore allow only two types of cells to be co-cultured at a time. However if the culture inserts are made smaller in sizes it would be possible to incorporate more than one in a single culture vessel.

Cell culture surface of culture inserts can be a dialysis membrane, ultrafiltration membrane, micro-filtration membrane or mesh. These cell culture surfaces made by a variety of membranes and mesh support the 3D scaffold for the cell culture. In addition to the physical support various membranes will have selective effects on the culture by their property to filter molecules of various sizes. For example, dialysis membrane will allow exchange of small solutes like nutrition, oxygen and toxic molecules between the culture vessel and the culture inserts. Similarly ultra filtration membrane will allow exchange of protein factors among culture inserts and the specific cells being cultured in them through the culture media in the culture vessel. Micro filtration membrane and meshes will allow free exchange of all types of molecules among culture inserts through the culture media.

Cell interactive biomaterial scaffold can be incorporated in the culture inserts during the culture or prior to culture.

In another aspect of this invention is to compartmentalize the culture vessel itself in more than one chamber, hence allowing cells from more than one tissue type or organ to be co-cultured in a single vessel. The compartmentalization can be achieved by semi-permeable membrane for selective ion exchange.

Removal of the spent medium and replenishment of fresh medium to supply nutrient to cells can be accomplished by manual or automatically at a regular interval or continuously. Provision of an inlet and outlet access to the culture medium connected through conduits to a medium reservoir and a spent medium collection chamber can accomplish removal and replenishment of cell culture media using gravitational flow, a peristaltic pump, ultrasound pump or any other type of fluid transfer devices.

In another aspect of this invention is to facilitate in vivo cell culture. Cells are transplanted for research or therapeutic purposes from donor to host. Providing in vivo like condition for cell culture is complicated and it is sometime advisable to study the behaviour of cells in most natural format by transplanting them in vivo. Cells may need be cultured for a few days in the 3D manner on a orous scaffold biomaterial before transplanting or directly transplanted along with biomaterial scaffold. Retrieval and analysis of cells is convenient if they are immobilized on porous scaffolds.

### EXAMPLES

### Example 1

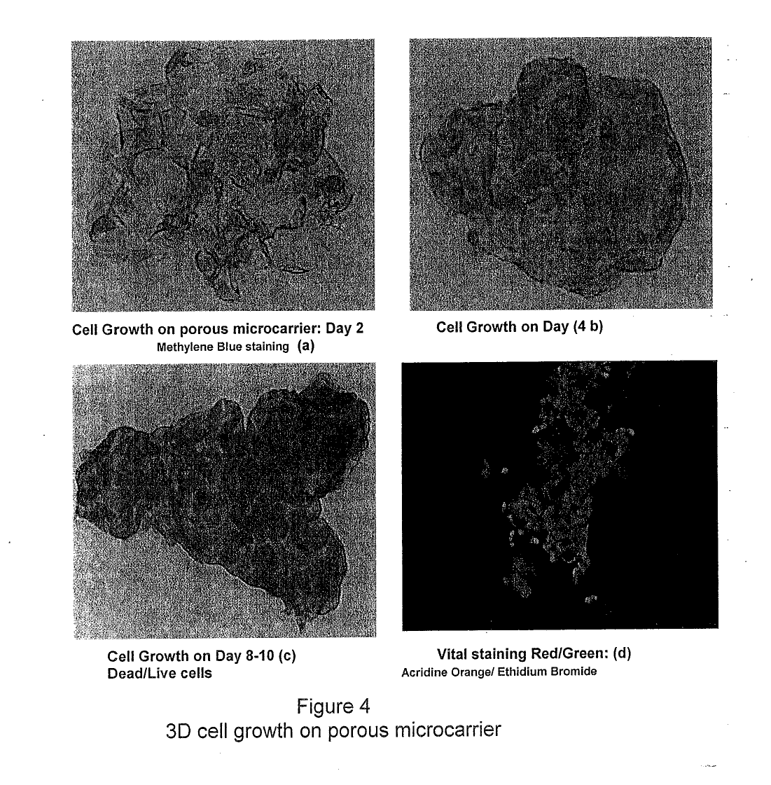

Porous microcarrier is prepared as per method provided in U.S. patent application Ser. No. 12/171,910. A Petri dish is coated with Poly hydroxyethyl methacrylate (Poly HEMA Sigma-Aldrich Co. P3932) to avoid cell attachment on the Petri dish surface. Coated Petri dishes are washed with water and sterilized by gamma irradiation or autoclaving. Microcarrier is suspended in phosphate buffered saline at a concentration of 2 mg/mL and autoclaved. Before culture microcarrier are washed with phosphate buffered saline and resuspended in complete cell culture medium (IMDM with 5% fetal calf serum) and plated in Poly HEMA coated Petri dishes. Vero cell a concentration of 80,000 cell/mL are inoculated in the Petri dish. Culture is incubated in 5% CO₂ at 37° C. for 15 days with regular change of medium every second day. A thick growth of cells in 3D is achieved as evident in the FIG. 4.

### Example 2

A thin sheet of glass (cover slip) or transparent plastic is coated with Poly HEMA as in example 1. Porous microcarrier is coated on a spot as densely as possible using medical grade silicon elastomer MDX 4-4210 by tapping gently porous microcarrier on to a thin layer spot of silicon elastomer. These glass or plastic cover slips are placed in Poly HEMA coated Petri dishes and sterilized by gamma irradiation. The cover slips are provided with a holding edge at an angel to the flat surface for easy retrieval.

Vero cells are suspended in complete medium as in example 1 and inoculated in the Petri dishes after washing with PBS two to three times.

Cells do not grow on the Poly HEMA coated surface and are confined to grow on the porous microcarrier coated area in a three dimensional manner. After the culture cover slip is retrieved from the Petri dish and observed under confocal microscope for viable cell count. (FIG. 4d).

### Example 3

Petri dishes are coated with Poly HEMA in the manner given in Example 1. Porous microcarrier is coated in the manner given in example 2 either as a single dot, multiple dots or individual porous microcarrier discrete particles. Sterilization can be performed by gamma irradiation or autoclaving. Shape of coated spot can be varied to identify them from one another during the culture.

Cell culture is performed as in example 2 and three dimensional growth is observed in the porous microcarrier coated spots. (FIG. 4).

### Example 4

Culture inserts are made by hollow poly styrene cylinders with open ends and closing one end either by a nylon mesh, a microfiltration membrane, a ultra filtration membrane or a dialysis membrane by sealing the mesh/membrane using medical grade adhesive, ultrasound welding or laser sealing methods. The diameter of cylinders may vary from 0.3 mm to 30 mm. These hollow cylinders with membrane element can be inserted in the Petri dish using a support to hang them from the rim or by the provision of leg support in more than one numbers.

### Example 5

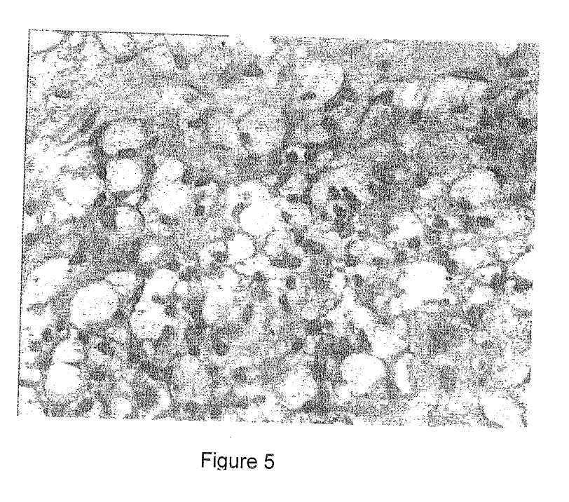

Primary rat liver cells are cultured on porous microcarrier in a Poly HEMA coated Petri dish. Young rats (species) are taken and liver cells isolated by the procedure described (Ref). Cells are cultured in DMEM with 10% fetal calf serum along with porous microcarrier of example 1. After 10 days microcarrier with growing cells are harvested and stained to observe under microscope (FIG. 5).

### Example 6

Cultured rat liver cells on porous microcarrier of example 5 are washed with DMEM medium without serum thrice and about 1 mL compact microcarrier is grafted in rat peritoneum cavity using a hypodermic syringe. Microcarrier are harvested form rat peritoneum after 14 days by sacrificing the rat and fixed and examined histologically. Formation of blood vessels has been observed deep inside the microcarrier supporting liver cells.

1-10. (canceled)

11. A device capable of providing artificial conditions for a three dimensional culture of animal and human cells comprising:
a first component comprised of a vessel coated in the entire internal surface rendered not suitable for cell adhesion and proliferation; and
a second component comprised of a cell interactive biomaterial on which cells can attach and grow as monolayer or multiple layers in three dimensions.

12. The device as claimed in claim 11, wherein the cell interactive biomaterial is proteins and glycans of extracellular origin isolated from natural tissues of animal organs, human organs, synthetically products, or any combination of natural and synthetic biomaterial either in the chemically bonded form or a simple admixture.

13. The device as claimed in claim 12, wherein the cell interactive biomaterial comprise single or multiple dots of either a thin surface coating and/or 3D porous scaffolds material of predefined compositions.

14. The device as claimed in claim 13, wherein said interactive biomaterial comprise of culture inserts floating or submerged into a cell culture medium.

15. The device as claimed in claim 14, wherein said interactive biomaterial comprise supports to hang them from a rim or legs for support.

16. The device as claimed in claim 14, wherein said culture inserts comprises of selective membrane of dialysis, ultra filtration, micro-filtration or meshes to allow selective or free exchange of molecules and cells amongst the islands and common nutrition medium.

17. The device as claimed in claim 11, wherein the device is divided into one or more chambers using semi-permeable membrane partitions.

18. The device as claimed in claim 11, further comprising an input and output port for nutrient media addition and removal during perfusion.

19. The device as claimed in claim 18, wherein the perfusion of culture medium is achieved by gravity flow by changing the level of cell culture medium, either by elevating or depressing the device of claim 11 once connected with a cell culture medium reservoir to inlet and outlet ports.

20. The device as claimed in claim 12, wherein the cell interactive biomaterial is used as biomaterial surface for in vivo cell culture as implants in animals.
